SEQUENCE LISTING (11 sequences)

SEQ ID NO: 1 - DNA, 23 bp, Artificial Sequence
  primer Brca2-11F
ctcatcatttgttgcctcacttc

SEQ ID NO: 2 - DNA, 21 bp, Artificial Sequence
  primer Brca2-11R
tgttggatacaaggcatgtac

SEQ ID NO: 3 - DNA, 20 bp, Artificial Sequence
  primer mTR-WT
gcactccttacaagggacga

SEQ ID NO: 4 - DNA, 20 bp, Artificial Sequence
  primer mTR-common
cttcaatttccttggcttcg

SEQ ID NO: 5 - DNA, 22 bp, Artificial Sequence
  primer mTR-mutant
atttgtcacgtcctgcacgacg

SEQ ID NO: 6 - DNA, 20 bp, Artificial Sequence
  primer CRE-3F
cggcatggtgcaagttgaat

SEQ ID NO: 7 - DNA, 20 bp, Artificial Sequence
  primer CRE-3R
cggtgctaaccagcgttttc

SEQ ID NO: 8 - DNA, 24 bp, Artificial Sequence
  primer CRE-internal-F
ctaggccacagaattgaaagatct

SEQ ID NO: 9 - DNA, 25 bp, Artificial Sequence
  primer CRE-internal-R
gtaggtggaaattctagcatcatcc

SEQ ID NO: 10 - DNA, 20 bp, Artificial Sequence
  primer BubR1K243R/+ F
gaggtaaaggcaggggaatc

SEQ ID NO: 11 - DNA, 20 bp, Artificial Sequence
  primer BubR1K243R/+ R
gagaaagcgggggtcattat

(19) United States
(12) Patent Application Publication
(10) Pub. No.: US 2019/0376043 A1
(43) Pub. Date: Dec. 12, 2019

(54) MUTANT MOUSE-DERIVED PANCREATIC ORGANOID AND USE THEREOF

(71) Applicant: Seoul National University R&DB Foundation, Seoul (KR)

(72) Inventors: Hyunsook Lee, Seoul (KR); Mi-Sun Kwon, Seoul (KR); Sangjin Paik, Seoul (KR); Jennifer Jaeun Lee, Seoul (KR); So Young Joo, Seoul (KR)

(21) Appl. No.: 16/448,966

(22) Filed: Jun. 21, 2019

Related U.S. Application Data
(63) Continuation of application No. PCT/KR2018/006722, filed on Jun. 14, 2018.

(30) Foreign Application Priority Data
Jun. 14, 2017 (KR) 10-2017-0074558

(51) Int. Cl. C12N 5/071; G01N 33/50; A01K 67/027

(52) U.S. Cl. CPC C12N 5/0677; G01N 33/507; A01K 2267/0331; A01K 2227/105; A01K 2267/025; A01K 67/0275

(57) ABSTRACT

Provided are a three-dimensional pancreatic organoid derived from the pancreas of a genetically modified mouse, a method for fabricating the three-dimensional pancreatic organoid, and use of the three-dimensional pancreatic organoid for drug effect verification and/or drug screening.

### TECHNICAL FIELD

There are provided a three-dimensional pancreatic organoid derived from the pancreas of a genetically modified mouse, a method for fabricating the three-dimensional pancreatic organoid, and a use of the three-dimensional pancreatic organoid for drug effect verification and/or drug screening.

### BACKGROUND ART

Most anticancer therapeutic agents developed through existing new drug development studies are not actively used in actual cancer patients due to their side effects or low efficacy in clinical practice. In addition, even patients with the same cancer often exhibit different responsiveness to the same anticancer chemotherapeutic agent, and thus there is a need to conduct studies for individual responsiveness of each patient.

Recently, completion of the human genome project has accumulated sequencing-related reference information for human genetic information (Lander E. S., Linton L. M., Birren B., Nusbaum C., Zody M. C., Baldwin J., et al., (2001). Initial sequencing and analysis of the human genome. Nature 409, 860-921. 10.1038/35057062). As a result, it is expected that whole genome sequencing (WGS) for humans will gradually reach an accessible level and be more actively used for cancer research in the future. However, information on cancer cell tissues derived from conventional two-dimensional cell culture methods does not accurately reflect situations in vivo, and thus there is an increasing demand for a new cell culture method. A technique of culturing a three-dimensional organoid is emerging as a technique capable of meeting such a demand. The three-dimensional organoid refers to cells that originate from stem cells and grow in a three-dimensional structure, the cells simulating a specific organ and forming themselves (Clevers, H. 2016. Modeling development and disease with organoids. Cell. 165: 1586-1597. http://dx.doi.org/10.1016/j.cell.2016.05.082). Such three-dimensional organoid is a system capable of modeling actual cancer more closely to its in vivo state due to genetic information being contained intact.

The most important thing in anticancer therapy research is an in vitro model and an in vivo animal model which simulate cancer closely to its actual state. The most basically used method for such cancer modeling is cell culture. Methods for continuously culturing cancer cell lines derived from mouse or human tissues in a monomolecular layer state and constructing the same so that observation and experiment can be carried out in vitro have been most actively used to date. However, this two-dimensional culture method has a disadvantage in that it does not reflect an in vivo situation where cells actually exist in a three-dimensional manner (Lee et al., 2008; Vunjak-Novakovic and Freed, 1998). On the other hand, cells cultured through a three-dimensional organoid culture method have genetic information which is almost similar to that of cancer cell tissues actually derived from patients (Dong Gao et al., 2014. Organoid Cultures Derived from Patients with Advanced Prostate Cancer, Cell, 176-187, https://doi.org/10.1016/j.cell.2014.08.016). Accordingly, it is expected that application studies using these three-dimensional organoids will form a basis for providing customized medical care to actual patients.

### TECHNICAL PROBLEM

In an aspect, there is provided a three-dimensional pancreatic organoid derived from the pancreas of a genetically modified mouse.

In another aspect, there is provided a method for fabricating a three-dimensional pancreatic organoid, comprising a step of performing three-dimensional culture of pancreatic cells of a genetically modified mouse.

In yet another aspect, there is provided a composition for verifying drug effect, comprising the three-dimensional pancreatic organoid derived from the pancreas of the genetically modified mouse.

In still yet another aspect, there is provided a method for verifying drug effect or providing information on drug responsiveness verification, comprising a step of treating, with a drug, the three-dimensional pancreatic organoid derived from the pancreas of the genetically modified mouse.

In still yet another aspect, there is provided a composition for drug screening, comprising the three-dimensional pancreatic organoid derived from the pancreas of the genetically modified mouse.

In still yet another aspect, there is provided a method for drug screening, comprising a step of treating, with a candidate substance, the three-dimensional pancreatic organoid derived from the pancreas of the genetically modified mouse.

In still yet another aspect, there is provided a use (or a method of use) of an organoid as a carcinogenesis model, comprising a step of karyotyping the three-dimensional pancreatic organoid derived from the pancreas of the genetically modified mouse and comparing a karyotype pattern of the organoid depending on genotype with a wild type.

In still yet another aspect, there is provided a method for identifying growth of an organoid depending on genotype, comprising a step of observing division rate and pattern of the three-dimensional pancreatic organoid derived from the pancreas of the genetically modified mouse.

### SOLUTION TO PROBLEM

The present invention aims at modeling cancer closely to its in vivo state, and usefully applying the same for drug efficacy verification depending on genetic modification, by using a gene analysis method such as WGS and organoids obtained from a normal tissue and a cancer tissue of a genetically modified mouse. The present invention intends to present a new method for verifying an anticancer chemotherapeutic agent, through treatment with a drug which complies with genotype, using WGS, and organoids derived from genetically engineered mice, human patients. In pancreatic organoids derived from genetically modified mice provided in the present specification, accumulation of genetic modifications in a carcinogenesis process can be compared by performing long-term culture of organoids having respective genotypes and identifying, through WGS, whether or not other genetic modifications are involved.

An object of the present invention is to provide a three-dimensional organoid derived from the pancreas of a genetically modified mouse, the organoid capable of simulating a biological tissue, in particular, a cancer tissue, which three-dimensionally exists in the living body, in a manner similar to the living body.

The organoid means a three-dimensional cell assembly produced, through self-renewal and self-organization, from adult stem cells (ASCs), embryonic stem cells (ESCs), induced pluripotent stem cells (iPSCs), or the like. Using a three-dimensional culture method, cells are agglomerated and recombined again to make an environment similar to that in the living body, which makes it possible to overcome limitations of 2-D cell lines cultured by a 2-D culture method and thus allows physiological activity functions in the living body to be similarly reproduced. Thus, the organoid can be applied to disease modeling, drug screening, and the like.

As used herein, a pancreatic organoid means a three-dimensional cell assembly obtained by culturing pancreatic cells (for example, pancreatic adult stem cells) with a three-dimensional culture method.

In an aspect, there is provided a three-dimensional pancreatic organoid derived from the pancreas of a genetically modified mouse.

In the present specification, the genetically modified mouse means a mouse having one or more modifications selected from the following modifications:

(1) modification in BRCA2 gene;

(2) knockout of telomerase RNA component (TERC); and

(3) genetic modification which induces modification of a residue for acetylation in BubR1 protein.

In an embodiment, the genetically modified mouse may carry:

(1) modification in BRCA2 gene,

(1) modification in BRCA2 gene and (2) knockout of telomerase RNA component (TERC), or

(3) genetic modification which induces modification of a residue for acetylation in BubR1 protein.

The BRCA2 (breast cancer 2) gene is a gene located on chromosome 5 (Chr 5: 150.52-150.57 Mb) of mice (Mus musculus) and may be selected from GebBank Accession No. NM_001081001.2 (NP_001074470.1-encoding gene), GebBank Accession No. NM_009765.3 (NP_033895.2-encoding gene), and the like. In the genetically modified mouse, the modification in BRCA2 gene may mean complete deletion of exon 11 in the BRCA2 gene. In an embodiment, the genetically modified mouse may be, but is not limited to, a mouse in which specific conditional deletion occurs, for example, a mouse in which Cre recombinase-mediated deletion of exon 11 is induced in an allele (for example, conditional deletion is induced by a tamoxifen-like compound such as 4-hydroxytamoxifen). For the purpose of inducing the Cre recombinase-mediated deletion of exon 11, the genetically modified mouse may be one in which the Cre recombinase gene has been introduced. For example, introduction of the Cre recombinase gene may be carried out by breeding of a mouse in which exon 11 of the BRCA2 gene has been deleted and a mouse into which the Cre recombinase gene has been inserted, introduction of a conventional vector (for example, an adenovirus vector) containing the Cre recombinase gene with a conventional method, and the like, but is not limited thereto. The Cre recombinase may be derived from Bacteriophage P 1 , and may be represented by UniProtKB P06956 (GenBank Accession No. YP_006472.1; coding gene (mRNA): CDS (436 . . . 1467) of NC_005856.1). However, the Cre recombinase is not limited thereto.

The telomerase RNA component (TERC) means non-coding RNA (ncRNA) present in eukaryotic cells which is an RNA component of telomerase (ribonucleoprotein) having telomere-elongating activity. The telomerase RNA component of mice (Mus musculus) may be RNA encoded by GebBank Accession No. NR_001579.1. In the genetically modified mouse, the knockout of the telomerase RNA component may mean deletion of one or more RNAs or substitution with a different base than the original base, in the full-length RNA sequence (397 nt) of the telomerase RNA component, and examples thereof may include knockout (deletion) of the full-length RNA sequence, replacement of the TERC gene using a targeting vector, and the like may be mentioned.

The BubR1 (mitotic checkpoint serine/threonine kinase; Bub1b) is a gene which encodes a kinase involved in spindle checkpoint action and chromosome segregation. The BubR1 is located at the kinetochore, and acts to inhibit anaphase-promoting complex/cyclosome (APC/C) and delay entry into anaphase. Spindle checkpoint dysfunction is observed in various cancers. The BubR1 gene of mice (Mus musculus) may be GebBank Accession No. NM_009773.3 (NP_033903.2-encoding gene). In the genetically modified mouse, the BubR1 gene may be modified to encode a BubR1 protein in which a residue for acetylation is substituted; and the residue for acetylation may be, for example, lysine (K) (K243) which is the 243^{rd} amino acid residue in the amino acid sequence of NP_033903.2. The modification may be modification that inhibits acetylation of the residue (for example, K243) for acetylation. In an embodiment, the modification of BubR1 gene may be done such that the BubR1 gene encodes a modified BubR1 protein whose K243 has been changed to arginine (R).

An organoid generated from the pancreas of the above-described genetically modified mouse has an excellent ability to simulate an in vivo environment as compared with a wild-type mouse (a mouse that does not have the above-mentioned modification), and in particular, can more similarly simulate an in vivo cancer environment. Thus, the organoid can be more advantageously applied to various drug effect (responsiveness) tests and/or drug screening for anticancer agents and the like.

In another aspect, there is provided a method for fabricating a three-dimensional pancreatic organoid, comprising a step of performing three-dimensional culture of pancreatic cells of a genetically modified mouse.

The genetically modified mouse is as described above.

The step of performing three-dimensional culture may include the following steps:

(i) a step of dissociating a pancreatic tissue isolated from the genetically modified mouse;

(ii) a step of collecting cell pellets containing ductal cells from the dissociated pancreatic tissue; and

(iii) a step of culturing the collected cell pellets with a biosubstrate material.

The step (step (i)) of dissociating the pancreatic tissue may be carried out by treating the pancreatic tissue with a dissociation solution. The dissociation solution may contain Hank's Balanced Salt Solution (HBSS) (for example, 1 ml to 10 ml, 1 ml to 5 ml, 3 ml to 10 ml, or 3 to 5 ml), collagenase (for example, collagenase P) (for example, 0.1 mg/ml to 5 mg/ml, 0.1 mg/ml to 3 mg/ml, 0.5 mg/ml to 5 mg/ml, 0.5 mg/ml to 5 mg/ml, 0.5 mg/ml to 2 mg/ml, or 0.8 mg/ml to 1.5 mg/ml), DNase (for example, DNase 1) (for example, 0.01 mg/ml to 1 mg/ml, 0.01 mg/ml to 0.5 mg/ml, 0.01 mg/ml to 0.2 mg/ml, 0.05 mg/ml to 1 mg/ml, 0.05 mg/ml to 0.5 mg/ml, 0.05 mg/ml to 0.2 mg/ml, or 0.08 mg/ml to 0.15 mg/ml), and the like. In an aspect, the dissociation solution may be used in an amount of 0.5 to 5 ml, 0.5 to 4 ml, 0.5 to 3 ml, 1 to 5 ml, 1 to 4 ml, or 1 to 3 ml with respect to the pancreatic tissue (Vpan=about 1.08 mg/mm³). However, the amount of the dissociation solution is not limited thereto, and may be appropriately regulated and used depending on an amount of the pancreatic tissue. At this time, in order to facilitate dissociation of the tissue, physical dissociation (for example, a step of performing fine cutting with scissors, a knife, or the like) and treatment with the dissociation solution may be carried out at the same time or at different times in any order.

The step (ii) of collecting the cell pellets containing ductal cells may include a step (ii-1) of isolating ductal cells from the dissociated pancreatic tissue, and a step (ii-2) of centrifuging the isolated ductal cells to collect cell pellets. The step (ii-1) of isolating ductal cells from the dissociated pancreatic tissue may include a step of filtering the dissociated pancreatic tissue obtained in the step (i), and separating and taking up ductal cells among the cells which have failed to pass. The filtration may be carried out using a conventional cell strainer having a pore size (for example, 50 μm to 200 μm, 50 μm to 170 μm, 50 μm to 150 μm, 50 μm to 120 μm, 80 μm to 200 μm, 80 μm to 170 μm, 80 μm to 150 μm, 80 μm to 120 μm, or 90 μm to 110 μm) which does not pass ductal cells. Separation of the duct cells may be carried out by conventional cell identification means such as microscopic observation. The step (ii-2) of collecting the cell pellets may include a step of centrifuging the ductal cells isolated in the step (ii-1), for example, at 1000 rpm to 2000 rpm, 1000 rpm to 1800 rpm, 1000 rpm to 1600 rpm, 1200 rpm to 2000 rpm, 1200 rpm to 1800 rpm, 1200 rpm to 1600 rpm, 1400 rpm to 2000 rpm, 1400 rpm to 1800 rpm, or 1400 rpm to 1600 rpm, for 1 to 20 minutes, 1 to 15 minutes, 1 to 12 minutes, 5 to 20 minutes, 5 to 15 minutes, 5 to 12 minutes, 8 to 20 minutes, 8 to 15 minutes, or 8 to 12 minutes, to collect pellets.

In the step (iii) of culturing the collected cell pellets with a biosubstrate material, the biosubstrate material may include one or more selected from the group consisting of Matrigel, extracellular matrix (ECM), Basement Membrane Extract (BME), hyaluronic acid, and the like. The Matrigel means a mixture of gelatin-like proteins secreted in Engelbreth-Holm-Swarm (EHS) mouse sarcoma cells. The extracellular matrix is an assembly of biopolymers containing molecules which are synthesized in cells, and secreted and accumulated outside the cells, and may include one or more selected from the group consisting of fibrotic proteins such as collagen and elastin, complex proteins such as glycosaminoglycan, cell adhesion proteins such as fibronectin and laminin, and the like.

The culture in the step (iii) may be performed under a conventional cell culture condition, for example, a condition of 35° C. to 36° C. and 8% to 12% CO2. In addition, the culture may be performed for 1 to 14 days, 1 to 10 days, 1 to 7 days, 3 to 14 days, 3 to 10 days, or 3 to 7 days before subculture, and then the subculture may be repeatedly performed once or twice, or more times, with 1 to 14 days, 1 to 10 days, 1 to 7 days, 3 to 14 days, 3 to 10 days, or 3 to 7 days per each subculture, so that the culture can be continuously performed for up to 12 months, 9 months, or 6 months. A medium to be used for the culture may contain, but is not limited to, one or more selected from the group consisting of Advanced DMEM/F-12 (Dulbecco's Modified Eagle Medium/Ham's F-12), penicillin/streptomycin, Hank's Balanced Salt Solution (HEPES), GlutaMAX, N-acetylcysteine, a conditioned medium (for example, Rspo 1-conditioned medium), nicotinamide, gastrin (for example, gastrin I), a growth factor (for example, EGF, FGF, and the like), Noggin, a Noggin-conditioned medium, and the like.

The above-described three-dimensional pancreatic organoid derived from the pancreas of the genetically modified mouse may be fabricated by the method for fabricating a three-dimensional pancreatic organoid.

In yet another aspect, there is provided a composition for verifying drug effect, comprising the three-dimensional pancreatic organoid derived from the pancreas of the genetically modified mouse. In still yet another aspect, there is provided a method for verifying drug effect or providing information on drug effect verification, comprising a step of treating, with a drug, the three-dimensional pancreatic organoid derived from the pancreas of the genetically modified mouse. The drug may be a drug for treating a pancreatic disease, for example, a drug for treating a pancreatic disease associated with modification in BRCA2, modification in telomerase RNA component (TERC), and/or modification in BubR1, as described above, for example, an anti-cancer agent for pancreatic cancer. In an embodiment, the drug may be, but is not limited to, one or more selected from the group consisting of histone deacetylase inhibitors (HDACi; for example, Trichostatin A (TSA), suberoylanilide hydroxamic acid (SAHA), LMK-235, FK-228 (Romidepsin), and the like), PARP-1 (Poly [ADP-ribose] polymerase 1) inhibitor (for example, Olaparib, Veliparib (ABT-888), Iniparib (BSI-201), and the like), polo-like kinase 1 (plk 1) inhibitor (for example, BI2536, Volasertib (BI 6727), Rigosertib (ON-01910), and the like), and the like. The drug effect means a biological action to be achieved by the drug and may mean an effect such as alleviation, palliation, and treatment of the pancreatic disease (for example, pancreatic cancer). In a case where the pancreatic disease is pancreatic cancer, the drug effect may mean alleviation, elimination, inhibition of progression or metastasis, and the like, of pancreatic cancer.

In a case where the size and/or the number of the three-dimensional pancreatic organoids is decreased at the time of being treated with a drug, it may be decided (or determined) that the drug has an effect on a pancreatic disease (for example, pancreatic cancer). Accordingly, the method for verification or providing information on verification may include, after the step of performing treatment with the drug, a step of measuring the size and/or the number of the three-dimensional pancreatic organoids, which have been treated with the drug, and comparing the measurement with three-dimensional pancreatic organoids which have not been treated with the drug. For this purpose, the comparing step may further include a step of measuring the size and/or number of the three-dimensional pancreatic organoids which have not been treated with the drug, and this step may be carried out, simultaneously with the step of measuring the size and/or number of the three-dimensional pancreatic organoids which have been treated with the drug, or the two steps may be carried out at different times in any order. The three-dimensional pancreatic organoids which have not been treated with the drug may be the three-dimensional pancreatic organoids before treatment with the drug, or some three-dimensional pancreatic organoids which are left after treating some of the three-dimensional pancreatic organoids with the drug. In addition, the method for verification or providing information on verification may include, after the comparing step, a step of deciding (or determining) that the drug has an effect on a pancreatic disease (for example, pancreatic cancer) in a case where the size and/or number of the three-dimensional pancreatic organoids which have been treated with the drug is decreased as compared with the three-dimensional pancreatic organoids which have not been treated with the drug.

In still yet another aspect, there is provided a composition for drug screening, comprising the three-dimensional pancreatic organoid derived from the pancreas of the genetically modified mouse.

In still yet another aspect, there is provided a method for drug screening, comprising a step of treating, with a candidate substance, the three-dimensional pancreatic organoid derived from the pancreas of the genetically modified mouse. The candidate substance may be selected among all bioactive substances, and may be, for example, one or more selected from the group consisting of bioactive small molecular chemicals, peptides, proteins (for example, antibodies, other protein drugs, and the like), nucleic acid molecules, extracts (for example, animal or plant extracts), and the like. The drug screening method may be for screening a drug having a therapeutic effect on a pancreatic disease. The pancreatic disease may be a pancreatic disease associated with modification in BRCA2, modification in telomerase RNA component (TERC), and/or modification in BubR1, as described above, for example, pancreatic cancer.

In a case where the size (or volume; hereinafter ‘size’ is used to have a meaning including volume) and/or number of the three-dimensional pancreatic organoids is decreased at the time of being treated with a candidate substance, it may be decided (or determined) that the candidate substance is a drug having an effect on a pancreatic disease (for example, pancreatic cancer). Thus, the screening method may include, after the step of performing treatment with a candidate substance, a step of measuring the size and/or number of the three-dimensional pancreatic organoids, which have been treated with the candidate substance, and comparing the measurement with three-dimensional pancreatic organoids which have not been treated with the candidate substance. For this purpose, the comparing step may further include a step of measuring the size and/or number of the three-dimensional pancreatic organoids which have not been treated with the candidate substance, and this step may be carried out, simultaneously with the step of measuring the size and/or number of the three-dimensional pancreatic organoids which have been treated with the candidate substance, or the two steps may be carried out at different times in any order. The three-dimensional pancreatic organoids which have not been treated with the candidate substance may be the three-dimensional pancreatic organoids before treatment with the candidate substance, or some three-dimensional pancreatic organoids which are left after treating some of the three-dimensional pancreatic organoids with the candidate substance. In addition, the screening method may further include, after the comparing step, a step of deciding (or determining) that the drug is a candidate drug having an effect on a pancreatic disease (for example, pancreatic cancer) in a case where the size and/or number of the three-dimensional pancreatic organoids which have been treated with the candidate substance is decreased as compared with the three-dimensional pancreatic organoids which have not been treated with the candidate substance.

In still yet another aspect, there is provided a use (or a method of use) of an organoid as a carcinogenesis model, comprising a step of karyotyping the three-dimensional pancreatic organoid derived from the pancreas of the genetically modified mouse and comparing a karyotype pattern of the organoid depending on genotype with a wild type.

In still yet another aspect, there is provided a method for identifying growth of an organoid depending on genotype, comprising a step of observing division rate and pattern of the three-dimensional pancreatic organoid derived from the pancreas of the genetically modified mouse.

### ADVANTAGEOUS EFFECTS OF INVENTION

The present invention provides a three-dimensional pancreatic organoid derived from the pancreas of a genetically modified mouse which has an excellent degree of biosimulation, a culture method therefor, and a use thereof. The three-dimensional pancreatic organoid can be usefully applied to drug efficacy (effect, responsiveness) verification and/or drug screening after treatment with a drug. In addition, the present invention can also be applied to karyotyping, observation for division rate and pattern of the organoid through genotyping thereof.

### BRIEF DESCRIPTION OF DRAWINGS

FIG. 1 illustrates images obtained by observing, through an inverted microscope (Zeiss), states in which pancreatic organoids derived from BRCA2 and/or TERC gene-deleted mutant mice are cultured.

FIG. 2A illustrates a photograph showing gel electrophoresis results which identify genetic modification (deletion of exon 11 in BRCA2 gene (Brca2^{F11/F11}), knockout of TERC gene (mTR^{-/-}), and insertion of CreER™ gene (CreER™) in organoids (M: DNA marker for band size discrimination, DW (distilled water): negative control of PCR, WT: wild-type, Brca2^{F11/F11}: band identifying that Brca2 exon 11 is flanked by loxP, mTR^{-/-}: band identifying that the TERC gene is knocked out, CreER™: band identifying that the Cre recombinase gene is inserted).

FIG. 2B illustrates gel electrophoresis results showing PCR products of the organoids identified as having BRCA2^{F11/F11} genotype, depending on presence or absence of treatment with 4-OHT, in which (−) is a result obtained in a case where treatment with 4-OHT is not performed, and (+) is a result obtained in a case where treatment with 4-OHT is performed to induce deficiency of exon 11 in BRCA2 gene.

FIG. 3 illustrates an image obtained by observing, through an inverted microscope (Zeiss), a result obtained by culturing pancreatic organoids derived from mutant mice which have been transgenic with K243R mutant of BubR1 gene.

FIG. 4 illustrates a photograph showing a gel electrophoresis result which identifies whether K243R mutant of BubR1 gene has been inserted in pancreatic organoids derived from genetically modified mice (BubR1^{K243R/+}) (M: DNA marker for band size discrimination, DW (distilled water): negative control of PCR).

FIG. 5 illustrates photographs showing states of pancreatic organoids derived from BRCA2 gene-deleted mice (Brca2^{F11/F11}; CreER™) which have been treated with histone deacetylase inhibitor (HDACi).

FIG. 6 illustrates photographs showing states of pancreatic organoids derived from BRCA2 gene-deleted mice (Brca2^{F11/F11}; CreER™) which have been treated with PARP-1 inhibitor or plk1 inhibitor alone or together with histone deacetylase inhibitor (HDACi).

FIG. 7 illustrates results obtained by observing, through live cell tracking using equipment of IncuCyte S3 Live-Cell Analysis System (Essen Bioscience), mouse pancreatic organoids having a wild-type gene for 24 hours after treatment with Trichostatin A (TSA) at 1 uM (scale bar: 2.1 mm).

FIG. 8 illustrates photographs showing a degree of growth for organoids, depending on presence or absence of treatment with 4-hydroxytamoxifen (4-OHT), the organoids being pancreatic organoids derived from Brca2^{F11/F11}; mTR^{-/-}; Cre-ER™ mice (G3) which have undergone three generations through breeding.

FIG. 9 illustrates fluorescence images showing an immunofluorescence analysis result for pancreatic organoids derived from BubR1^{K243R/+} mice. The left image shows a result for pancreatic organoids derived from wild-type mice, and the right image shows a result for pancreatic organoids derived from BubR1^{K243R/+} mice.

### DETAILED DESCRIPTION OF INVENTION

Hereinafter, the present invention will be described in more detail with reference to examples. However, these examples are merely illustrative and are not intended to limit the scope of the present invention. It will be apparent to those skilled in the art that the examples as described below can be modified within the scope that does not depart from the essential spirit of the invention.

### EXAMPLE 1

### Construction of Pancreatic Organoids Derived from Transgenic Mice

Mice (Brca2^{F11/Ff11}; exon 11-deleted) in which loxP had been located to flank a position of exon 11 in BRCA2 gene through Cre-loxP recombination using bacteriophage P1 so that conditional induction of defective BRCA2 is possible using CreER recombinase (Jos Jonkers, Ralph Meuwissen, Hanneke van der Gulden, Hans Peterse, Martin van der Valk and Anton Berns. 2001. Synergistic tumor suppressor activity of BRCA2 and p53 in a conditional mouse model for breast cancer. Nature Genetics. Vol. 29, pages 418-425), mice (mTR^{-/-}) in which telomerase RNA component (TERC) gene had been knocked out to lose activity of telomerase, and CMV-Cre mice (CreER™) with CMV promoter were prepared.

1.1. Construction of Targeting Vector for Exon of BRCA2

In order to target BRCA2 locus, a 13.5-kb lambda phage clone containing exons 8-12 of mouse BRCA2 gene (NM_001081001.2) was isolated from the genomic 129/Sv library (Agilent technologies). The phage inset was excised with NotI and subcloned into pGEM5 (Promega). Then, loxP-PGKneor-PGKtk-loxP dual selection cassette (Thermo Fisher Scientific) was inserted into AvrII position in intron 11 of the mouse BRCA2 gene, and a single loxP site was inserted into NspV position in intron 10 in direct orientation with the floxed selection cassette.

1.2. Construction of Targeting Vector for Telomerase RNA Component (TERC) gene

Mice in which the telomerase RNA component (TERC) gene (GebBank Accession No. NR_001579.1) has been completely deleted were obtained from Jackson lab (Stock No: 004132, B6.Cg-Terctm1Rdp/J) and used for the following experiments.

1.3. Production of Mice (Brca2^{F11/F11}; CreER™) in which Exon 11 of BRCA2 has been Deleted

The vector prepared in Example 1.1 above was isolated and purified, and introduced, by electroporation, into 129/Ola-derived mouse embryonic stem cells (ES cells) of E11 subclone IB10 (The Netherlands Cancer Institute). Colonies in which floxed selection marker and loxP site had been precisely inserted were selected by Southern blot. A mixture obtained by mixing Cre-expressing plasmids pOG231 (Addgene) and PGKpuro (Addgene) at a molar ratio of 10:1 was introduced into the embryonic stem cells by electroporation, to transiently induce Cre recombinase activity and puromycin resistance. 20 hours after electroporation, puromycin (1.8 μg/ml) was added to the medium and cultured for 48 hours. Dead cells were removed and surviving embryonic stem cells were further cultured in a non-selective medium for 10 days. The obtained embryonic stem cell clones were analyzed by Southern blotting to identify successful deletion of floxed marker, insertion of single loxP site, and generation of a conditional allele.

The obtained 12 to 15 mutant 129/Ola embryonic stem cells were injected into C57B1/6 blastocysts to generate germline chimera, which was bred with FVB/N mice (Jackson Laboratory) to produce outbred heterozygous offspring (BRCA2 conditional mutants). The obtained BRCA2 conditional mutants were bred with Cre mice (CMV-Cre mice (CreER™) with CMV promoter; Jackson Laboratory, Stock No. 004847) so that Cre-mediated deletion of floxed allele in the germline was performed, thereby producing mice (designated by Brca2^{F11/F11}; CreER™ or Brca2^{F11/F11}; mTR^{+/+}; CreER™) in which BRCA2 is conditionally deleted.

1.4. Production of Mice (Brca2F¹¹/F¹¹; mTR^{-/-}; CreER™) in which exon 11 of BRCA2 has been Deleted and TERC Gene has been Knocked Out

As in Example 1.3 above, the Brca2^{F11/F11}; CreER™ mice were obtained from the offspring produced by breeding the Brca2^{F11/F11} mice with the CreER™ mice. These Brca2^{F11/F11}; CreER™ mice were bred with the mTR^{-/+} mice (Example 1.2) to obtain offspring. From the obtained offspring, Brca2^{F11/F11}; CreER™; mTR^{-/+} mice were obtained, and bred with each other to obtain Brca2^{F11/F11}; CreER™; mTR^{-/-} mice with a Mendelian probability.

1.5. Production of Mice (BubR1^{K243R/+}) which have been Transgenic with K243R Mutant of BubR1 Gene

BubR1 gene which had been mutated, through site-directed mutagenesis, to induce substitution (K243R) of the 243^{rd} lysine (K) with arginine (R) in BubR1 (GenBank NP_033903.2; coding gene: NM_009773.3) was inserted into 129/Sv embryonic stem (ES) cells (Agilent Technologies) using pBluescript KS (+) vector (Addgene). The resultant was injected into blastocysts of C57BL/6 mice, to obtain heterologous mutant mice (BubR1^{K243R/+}) which had been transgenic with a mutant gene that encodes BubR1 having K243R mutation (see Inai Park et al. 2013. Loss of BubR1 acetylation causes defects in spindle assembly checkpoint signaling and promotes tumor formation. Journal of Cell Biology. 202 (2):295).

### EXAMPLE 2

### Fabrication of Pancreatic Organoids Derived from Mutant Mice in which BRCA2 Gene and/or TERC Gene have been Deleted

Pancreatic organoids were fabricated from respective mutant mice produced in Examples 1.3 and 1.4 above by the methods as described below:

Each mouse was euthanized and then dissected to obtain a pancreatic tissue. As a dissociation solution, a mixture obtained by performing mixing of 3 to 5 ml of Hank's Balanced Salt Solution (HBSS; GIBCO®), 1 mg/ml of collagenase P (Roche), and 0.1 mg/ml of DNase 1 (Sigma Aldrich) was prepared by being warmed in water bath at 37° C. The obtained entire mouse pancreatic tissue (Vpan=1.08 mg/mm³) was transferred to a 100 mm Petri dish. Then, 100 ul of the prepared dissociation solution was added thereto, and the tissue was finely cut 5 to 10 times using scissors or a knife. The finely cut pancreatic tissue (Vpan=1.08 mg/mm³) was transferred to a 50 ml conical tube, to which 3 to 5 ml of the dissolution solution was added. The tube was incubated by being placed in a shaking incubator at 230 rpm and 37° C. After 10 to 15 minutes, the tube was removed and 10 to 15 ml of cold FBS was added thereto. The obtained dissociated pancreatic tissue was passed through a 100 μm cell strainer and washed 2 to 3 times with HBSS. The pancreatic tissue, which did not pass through the cell strainer, was observed under a microscope, and ductal cells were chosen and picked. A process, in which the picked ductal cells were centrifuged at 1500 rpm for 10 minutes to collect pellets and then washing with HBSS was performed, was repeated twice.

The obtained cell pellets and 200 ul of Matrigel (Corning) were mixed so that a volume ratio of the cell pellets and the Matrigel was 1:5, and then respectively seeded into a 12-well or 24-well plate in an amount (of 100 to150 ul based on the 12-well plate) per well. About one hour after seeding, when the Matrigel had hardened, a medium was added in an amount of 1 ml based on the 12-well plate or 500 ul based on the 24-well plate, and incubated for 48 to 72 hours or longer in an incubator at 37° C. and 10% CO₂. The culture medium used at this time had the following composition: A mixture of Advanced DMEM/F-12 (Dulbecco's Modified Eagle Medium/Ham's F-12; Thermo Fisher Scientific) with 1% (vol/vol) penicillin/streptomycin, 10 mM HEPES, 1% GlutaMAX, 1:50 B27 supplement (Gibco), 1 mM N-acetylcysteine, 5% (vol/vol) Rspo 1-conditioned medium (Hans Clevers lab), 10 mM nicotinamide, 10 nM recombinant human [Leu15]-gastrin I (Sigma Aldrich), 50 ng/ml of recombinant mouse EGF (Peptron), 100 ng/ml of recombinant human FGF10 (Peptron), and 25 ng/ml of recombinant human Noggin (Peptron) or 5% (vol/vol) Noggin-conditioned medium (Hans Clevers lab).

In order to induce deletion of the BRCA2 gene and/or the TERC gene, the organoids were treated with 4-hydroxytamoxifen (4-OHT). Specifically, 4-hydroxytamoxifen (4-OHT) was dissolved in the organoid culture to be at 400 nM and cultured for 3 weeks or longer, starting from a time point, at which 24 to 48 hours lapsed after the pancreas was initially isolated from the mice and then culture thereof into organoid was constructed, until immediately before a time point at which treatment with a drug was performed for the drug screening as described below (see Examples 4 and 5).

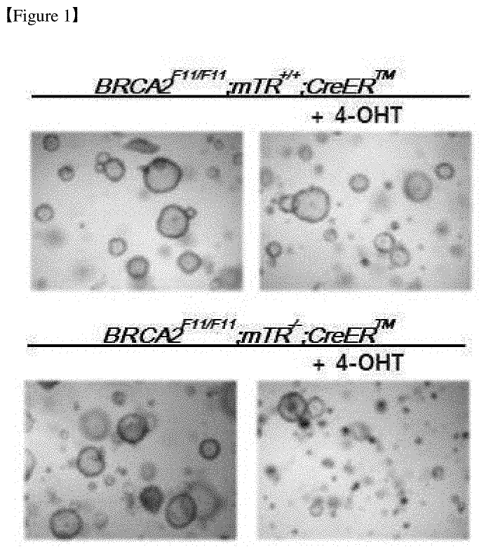

The results obtained by observing, with an inverted microscope (Zeiss), results caused by the above-described culture of the pancreatic organoids are illustrated in FIG. 1. As illustrated in FIG. 1, it can be identified that pancreatic organoids were successfully produced regardless of whether the BRCA2 gene and/or the TERC gene is deleted (whether treatment with 4-OHT is performed). It can be identified that the BRCA2 gene-deficient organoids were somewhat small in shape, but grew in a similar pattern to a wild type as the culture gradually proceeded.

Genomic PCR DNA gel electrophoresis was performed on the genomic DNA extracted by lysis of the obtained pancreatic organoids, so that genotype of the organoids was identified. Specifically, PCR was performed by repeating 30 cycles, each cycle including denaturation at 95° C. for 1 minute, annealing at 55° C. for 30 seconds, and elongation at 72° C. for 1 minute; and for electrophoresis, 5 ul of PCT product was mixed with 5 ul of Bromophenol or xylene on 1% (w/v) agarose gel and electrophoresed at 100 mV. The primers used at this time were as follows:

TABLE 1

| | | SEQ |
|---|---|---|
| | | ID |
| Primer | Nucleic acid sequence (5′→3′) | NO |
| Brca2-11F | CTCATCATTTGTTGCCTCACTTC | 1 |
| Brca2-11R | TGTTGGATACAAGGCATGTAC | 2 |
| mTR-WT | GCACTCCTTACAAGGGACGA | 3 |
| mTR-common | CTTCAATTTCCTTGGCTTCG | 4 |
| mTR-mutant | ATTTGTCACGTCCTGCACGACG | 5 |
| CRE-3F | CGGCATGGTGCAAGTTGAAT | 6 |
| CRE-3R | CGGTGCTAACCAGCGTTTTC | 7 |
| CRE- | CTAGGCCACAGAATTGAAAGATCT | 8 |
| internal-F | | |
| CRE- | GTAGGTGGAAATTCTAGCATCATCC | 9 |
| internal-R | | |

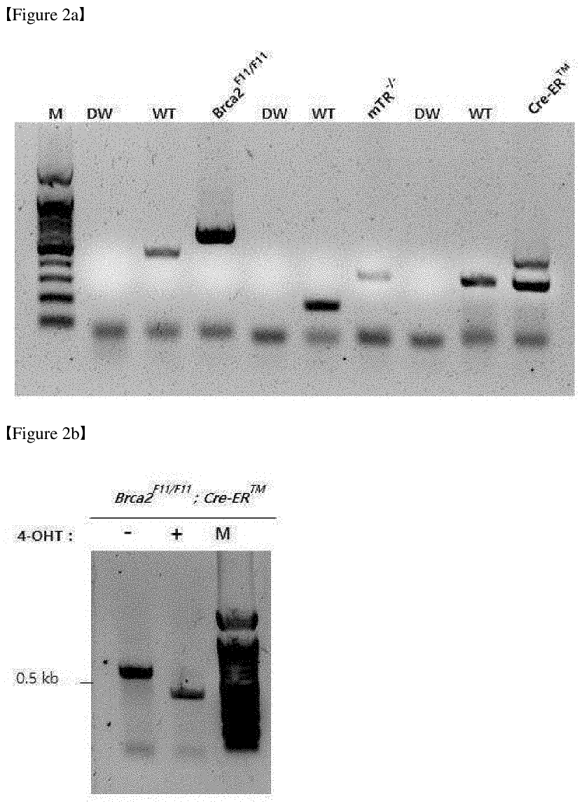

The obtained results are illustrated in FIGS. 2A and 2B. FIG. 2A illustrates a photograph showing gel electrophoresis results which identify genetic modification (deletion of exon 11 in BRCA2 gene) (Brca2^{F11/F11}), knockout of TERC gene (mTR^{-/-}), and insertion of CreER™ gene (CreER™) in organoids (M: DNA marker for band size discrimination, DW: negative control of PCR, WT: wild-type, Brca2^{F11/F11}: band identifying that Brca2 exon 11 is flanked by loxP, mTR^{-/-}: band identifying that the TERC gene is knocked out, CreER™: band identifying that the Cre recombinase gene is inserted).

FIG. 2B illustrates gel electrophoresis results showing PCR products of the organoids identified as having BRCA2^{F11/F11} genotype in FIG. 2A, depending on presence or absence of treatment with 4-OHT, in which (-) is a result obtained in a case where treatment with 4-OHT is not performed, and (+) is a result obtained in a case where treatment with 4-OHT is performed to induce deficiency of exon 11 in BRCA2 gene. As illustrated in FIG. 2B, it can be identified that in a case where treatment with 4-OHT was performed, a length of the PCR products became shorter than that in a case where treatment with 4-OHT was not performed, indicating deficiency of exon 11 in the BRCA2 gene.

### EXAMPLE 3

### Fabrication of Pancreatic Organoids Derived from Heterologous Mutant Mice which have been Transgenic with K243R Mutant of BubR1 Gene

With reference to the method in Example 2, pancreatic organoids were fabricated from the heterologous mutant mice (BubR1^{K243R/+}) which had been produced in Example 1.5 above and in which the mutant of BubR1 gene inducing K243R mutation had been knocked in. For comparison, pancreatic organoids derived from wild-type mice were fabricated with reference to Example 2.

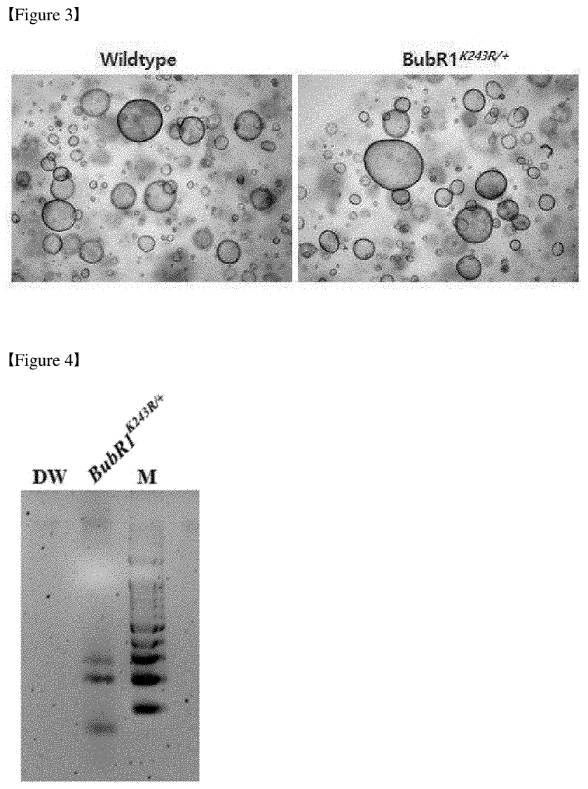

The results obtained by observing the obtained pancreatic organoids with an inverted microscope (Zeiss) are illustrated in FIG. 3. As illustrated in FIG. 3, it can be identified that pancreatic organoids can be successfully produced from the mutant mice (BubR1^{K243R/+}), as in the wild-type mice.

Genomic PCR DNA gel electrophoresis was performed on the genomic DNA extracted by lysis of the obtained pancreatic organoids, so that genotype of the organoids was identified. Specifically, PCR was performed by repeating 30 cycles, each cycle including denaturation at 95° C. for 1 minute, annealing at 55° C. for 30 seconds, and elongation at 72° C. for 1 minute; and for electrophoresis, 5 ul of PCT product was mixed with 5 ul of Bromophenol or xylene on 1% (w/v) agarose gel and electrophoresed at 100 mV. The primers used at this time were as follows:

TABLE 2

| | | SEQ |
|---|---|---|
| | Nucleic | ID |
| Primer | acid sequence (5′-3′) | NO |
| BubR1K243R/+ F | GAGGTAAAGGCAGGGGAATC | 10 |
| BubR1K243R/+ R | GAGAAAGCGGGGGTCATTAT | 11 |

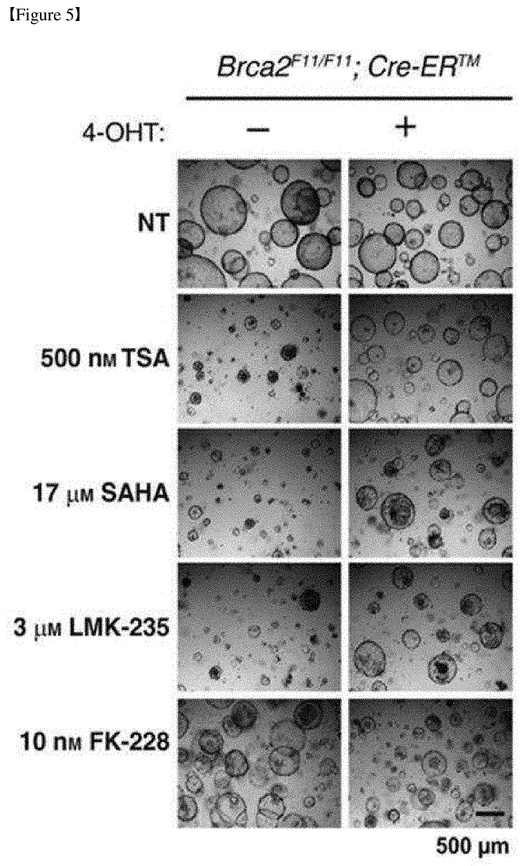

The obtained results are illustrated in FIG. 4. FIG. 4 illustrates a photograph showing a gel electrophoresis result which identifies whether K243R mutant of BubR1 gene has been inserted in pancreatic organoids derived from genetically modified mice (BubR1^{K243R/+}) (M: DNA marker for band size discrimination, DW: negative control of PCR). As identified in the lane indicated as BubR1^{K243R/+} in FIG. 4, bands at 145 bp and 219 bp are observed, which means that knock-in of the gene in question has successfully been achieved.

### EXAMPLE 4

### Drug Responsiveness Test of Pancreatic Organoids from Mutant Mice

With reference to the method in Example 2 above, pancreatic organoids derived from BRCA2 gene-deleted mice (Brca2^{F11/F11}; CreER™) were prepared. 4-hydroxytamoxifen (4-OHT) was added thereto in an amount of 400 nM, and the resultant was cultured for 3 weeks or longer so that partial deletion (deletion of exon 11) in BRCA2 gene was induced (see Example 2). For comparison, organoids (with normal BRCA2 gene) for which treatment with 4-OHT had not been performed was also prepared.

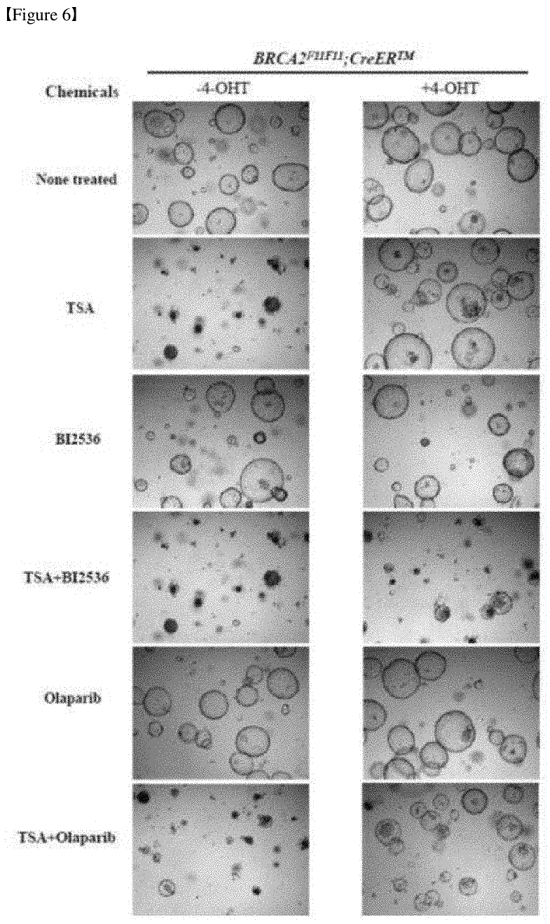

Electrophoresis results which identify BRCA2 deletion in pancreatic organoids derived from BRCA2 gene-deleted mice (Brca2^{F11/F11}; CreER™) are illustrated in FIG. 5A.

Organoids with normal BRCA2 gene (for which treatment with 4-OHT had not been performed) and organoids in which BRCA2 had been partially deleted (exon 11-deleted) were seeded in the same amount into 24-well plates. On the next day after seeding, the organoids were treated with a mixture of histone deacetylase inhibitor (HDACi), an anticancer agent, with a culture medium (see Example 2). Types and treatment concentrations of HDACi used at this time are shown in Table 3 below:

TABLE 3

| Type of HDACi | Treatment concentration |
|---|---|
| Trichostatin A (TSA) | 1 uM |
| Suberoylanilide hydroxamic | 17 uM |
| acid (SAHA) | |
| LMK-235 | 3 uM |
| FK-228, Romidepsin | 10 nM |

At this time, treatment with HDACi and treatment with 4-OHT were not performed simultaneously for pancreatic organoids in which BRCA2 had already been knocked out. 3 days after treatment (primary treatment) with HDACi, the culture medium was replaced with a fresh medium, and treatment (secondary treatment) with each HDACi was performed again at the concentration indicated in Table 3. Here, the primary treatment with HDACi was performed about 24 to 48 hours after seeding of the organoids, and 3 days after that, the second treatment was performed. At 6 days after the primary treatment with HDACi, a state of the organoids for which the treatment with HDACi had been performed was observed using an inverted microscope.

The obtained results are illustrated in FIG. 5. As illustrated in FIG. 5, it was observed that responsiveness to the tested drugs differs depending on the presence or absence of the BRCA2 gene. Through this observation, it can be identified that the Brca2^{F11/F11} mouse pancreatic organoid can be usefully used for identification of effects of a drug related to the BRCA2 gene and/or for drug screening.

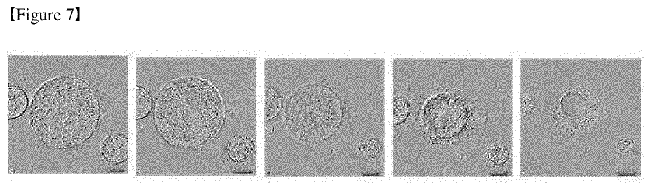

In addition, using the above-described method, states of the organoids obtained in cases of being treated with Olaparib (10 uM), a PARP-1 inhibitor, or BI2536 (10 nM), a plk 1 inhibitor, alone or in combination with HDACi (TSA 200 nM) were observed, and the results are illustrated in FIG. 6. As illustrated in FIG. 6, it was observed that responsiveness to the tested drugs differs depending on the presence or absence of the BRCA2 gene. Through this observation, it can be identified that the Brca2^{F11/F11} mouse pancreatic organoid can be usefully used for identification of effects of a drug related to the BRCA2 gene and/or for drug screening.

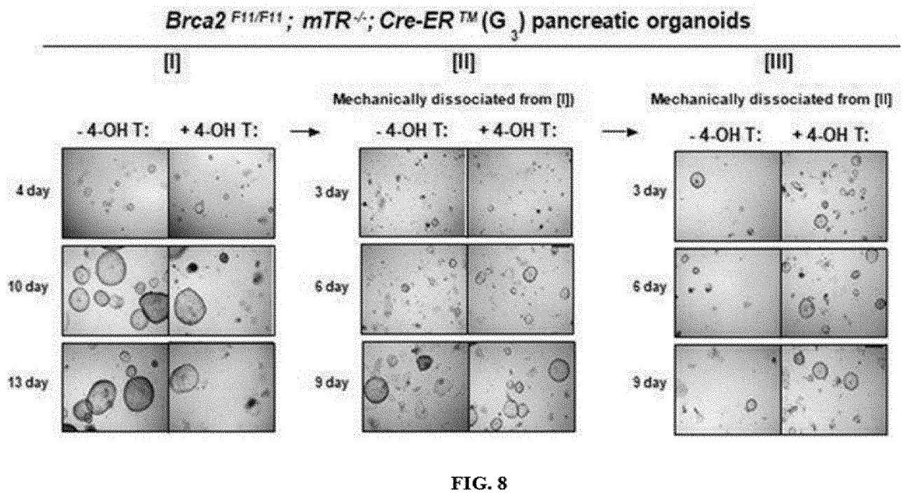

In addition, the results obtained by observing, through live cell tracking using equipment of IncuCyte S3 Live-Cell Analysis System (Essen Bioscience), mouse pancreatic organoids having a wild-type gene for 24 hours after treatment with Trichostatin A (TSA) at 1 uM are illustrated in FIG. 7. Respective images are images taken on a 4-hour basis. Scale bar represents 2.1 mm.

### EXAMPLE 5

### Construction of Model, which Simulates Carcinogenesis caused by Alternative Telomere Maintenance Mechanism (Alternative Lengthening of Telomeres (ALT)), by Continuous Culture of Brca2^{F11/F11}; mTR^{-/-}; Cre-ER™ Mouse Pancreatic Organoids

A cancer model simulating mechanism of alternative lengthening of telomeres (ALT) which maintains a length of telomeres without telomerase and causes continuous division was constructed through Brca2^{F11/F11}; mTR^{-/-}; Cre-ER™ generation 3 (G3) mouse pancreatic organoids which had been obtained, through three generations, by breeding of Brca2^{F11/F11}; mTk^{-/-}; Cre-ER™ mice with each other. In order to overcome a disadvantage of two-dimensionally cultured ALT cell lines used in existing ALT studies, that is, a disadvantage that genomic mutations have already accumulated and all environmental characteristics in the body are not reflected, ALT-induced mice (G3) were produced through three-way breeding of Brca2F11/F11; mTR−/−-; Cre-ER™ mice. In addition, pancreas-derived organoids which can simulate an in-vivo environment of the organ in the mice in question and allows direct identification of whether growth is inhibited were constructed, and it was identified that actual ALT is induced, and thus inhibition of growth due to deficiency of telomerase is overcome.

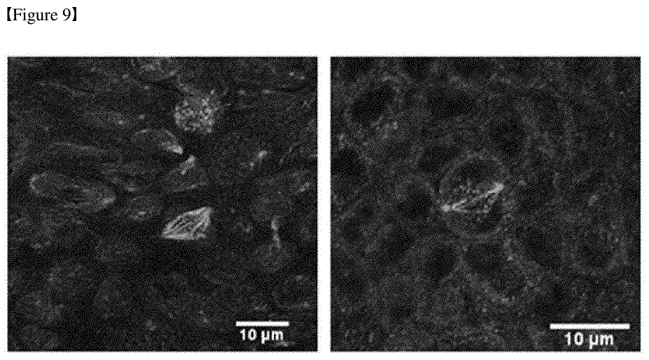

For construction of the ALT model of mouse pancreatic organoids, with reference to Example 2, pancreatic organoids were constructed from Brca2^{F11/F11}; mTk^{-/-}; Cre-ER™ mice which had undergone three generations (G3) through breeding. Then, treatment with tamoxifen (4-OHT) was performed or not performed so that Brca2 deficiency was induced or not induced. A treatment amount of 4-OHT was set as 400 nM. The pancreatic organoids were seeded on plates and the treatment was performed once every 2 days from one day after seeding the pancreatic organoids on the plates until treatment with a drug. Then, the organoids were observed with an inverted microscope (Zeiss) once every three days, and the results are illustrated in FIG. 8. Subculture was performed on the 10^{th} day in the order of [I], [II], and [III] illustrated in FIG. 8, and a degree of growth thereof was compared.

In [I], it was identified that the organoids (+4-OHT) which are deficient in both BRCA2 and TERC exhibit somewhat slower growth than the organoids (−4-OHT) which are deficient in only TERC.

In [II] (mechanically dissociated from [I]) , it was identified that the organoids (−4-OHT) which are deficient in only TERC exhibit inhibited growth as compared with the previous subculture, and that the organoids (+4-OHT) which are deficient in both BRCA2 and TERC exhibit slow growth as compared with the previous subculture.

In [III] (mechanically dissociated from [II]), it was identified that the organoids (−4-OHT) which are deficient in only TERC exhibit greatly decreased growth and are in a state where no more sustainable culture is possible, and that the organoids (+4-OHT) which are deficient in both BRCA2 and TERC are in a state where sustainable culture is possible. From these results, it is identified that organoids in which an ALT mechanism is activated and carcinogenesis proceeds were cultured.

### EXAMPLE 6

### Imaging on BubR1^{K243R/+} Mouse Pancreatic Organoids through Immunofluorescence Assay (IFA)

With reference to Examples 2 and 3, the BubR1^{K243R/+} mouse pancreatic organoids were cultured, and then staining with DAPI (blue), Tublin (green), and BubR1 (red) was performed, through which it was identified that the constructed organoids can be utilized as a new model to study genome instability.

First, a process, in which the cultured organoids are transferred to a 15 ml conical tube, the tube is filled with cold PBS up to 15 ml, centrifugation is performed at 1200 rpm for 5 minutes, and washing with PBS is performed, was repeated three times to remove Matrigel. The cells were fixed by adding 4% (w/v) paraformaldehyde (PFA), incubated for 30 minutes, and then washed three times with PBS. Triton X-100 solution (in PBS; concentration: 1% (v/v)) was added to the cell sample and incubation was performed for 1 hour. Then, Triton X-100 solution (in PBS; concentration: 2% (v/v)) was added thereto and washing was performed twice. Then, blocking buffer (0.279 g of BSA, 450 ul of goat serum, 180 ul of Triton X-100, 450 ul of PBS 20X, 7920 ul of DW) was added thereto and incubation was performed for 30 minutes. Treatment with BubR1 antibody (BD Biosciences) was performed and incubation was performed for 16 hours. Washing with 0.2% (v/v) PBS-T (Triton X-100 solution in PBS) was performed three times for 20 minutes each time. HRP-tagged secondary antibody (Thermo Fisher Scientific) was added thereto and incubation was performed for 16 hours. Washing with 0.2% (v/v) PBS-T was performed three times for 20 minutes each time, and treatment with FITC-conjugated Tublin antibody (Abcam) was performed at a ratio of 1:1000. Incubation was performed for 2 days. Washing with 0.2% (v/v) PBS-T was performed three times for 20 minutes each time. Then, DAPI was added thereto and incubation was performed for one day. After removing the supernatant, the resultant was transfer to an 8-well chamber and fluorescence images were observed using a confocal microscope.

The observed fluorescence images are illustrated in FIG. 9. In FIG. 9, the left image shows a result for pancreatic organoids derived from wild-type mice, and the right image shows a result for pancreatic organoids derived from BubR1^{K243R/+}mice.

1.-16. (canceled)

17. A method for screening a candidate substance for treating a pancreatic disease, comprising:
(a) a step of preparing a three-dimensional pancreatic organoid derived from a genetically modified mouse, the organoid being derived from the pancreas of the genetically modified mouse, wherein the genetically modified mouse has a modification which is deletion of exon 11 in BRCA2 gene; and
(b) a step of treating the organoid with a candidate substance for treating a pancreatic disease.

18. The method according to claim 17, further comprising:
(c) a step of determining the candidate substance as a drug for treating a pancreatic disease which is resistant to a histone deacetylase inhibitor drug, in a case where the candidate substance has a therapeutic effect for a pancreatic disease.

19. The method according to claim 17, wherein the pancreatic disease is pancreatic cancer.

20. A pancreatic organoid in which BRCA2 is conditionally deleted, the organoid being derived from a pancreatic tissue of a mouse which carries a loxP cassette and a Cre-expressing plasmid such that exon 11 in BRCA2 gene is conditionally deleted.

21. The pancreatic organoid according to claim 20, wherein the organoid has disease of pancreatic cancer.

22. The pancreatic organoid according to claim 20, wherein the organoid is for screening a candidate therapeutic substance for treating a pancreatic disease which is resistant to a histone deacetylase inhibitor drug.

23. A method for fabricating the pancreatic organoid according to claim 20, comprising:
(a) a step of constructing a vector in which a loxP cassette is inserted into a position of intron 11 in BRCA2 gene;
(b) a step of introducing the vector and a Cre-expressing plasmid into mouse embryonic stem cells (ES cells);
(c) a step of producing a BRCA2 conditional mutant mouse from a mouse developed from the mouse embryonic stem cells;
(d) a step of breeding the BRCA2 conditional mutant mouse with a Cre mouse (CreER™), to produce a mouse (Brca2F11/F11; CreER™) in which BRCA2 is conditionally deleted; and
(e) a step of fabricating an organoid from a pancreatic tissue of the mouse in which BRCA2 is conditionally deleted.

24. The method according to claim 23, wherein the organoid has disease of pancreatic cancer.

25. The method according to claim 23, wherein the organoid is for screening a candidate therapeutic substance for treating a pancreatic disease which is resistant to a histone deacetylase inhibitor drug.
